(19) United States
(12) Patent Application Publication
(10) Pub. No.: US 2011/0244551 A1
(43) Pub. Date: Oct. 6, 2011

(54) EXTRACT HAVING PROTEASE ACTIVITY

(75) Inventors: Mohd Yazid abdul Manap, Selangor (MY); Abdullah Sipat, Selangor (MY); Yousif Mohamed Ahmed Idris, Khartoum North (SD); Nassim Naderi, Selangor (MY)

(73) Assignee: UNIVERSITI PUTRA MALAYSIA, Selangor (MY)

(21) Appl. No.: 13/129,216

(22) PCT Filed: Nov. 16, 2009
(86) PCT No.: PCT/MY09/00195

§ 371 Date: Jun. 28, 2011

(30) Foreign Application Priority Data
Nov. 14, 2008 (MY) PI 20084588

(51) Int. Cl. C12N 9/48

(52) U.S. Cl. 435/212

(57) ABSTRACT

The present invention discloses a composition comprising a proteinaceous extract of Streblus asper having substantially protease activity that degrades proteins by hydrolysis of peptide bonds. The proteinaceous extract of Streblus asper according to the present invention is suitable for us as a meat quality-improving agent and a milk coagulant in food processing industries, as well as an additive in the manufacture of detergents.

The present invention relates generally to proteinaceous compositions. More particularly, the present invention relates to a composition comprising proteinaceous extracts of Streblus asper that is substantially having protease activity.

### BACKGROUND TO THE INVENTION

Proteases are enzymes that degrade proteins by hydrolysis of peptide bonds. Practical uses of proteolytic enzymes are in medicine, softening of leather, laundry detergents and food processing. In food industry protease are being used in baked goods, beer and wine, cereals, milk, meat tenderization, fish products, legumes and for production of protein hydrolysates and flavour extracts.

Among the proteases used in food processing are the milk-clotting enzymes for cheese production. In order for milk to coagulate and eventually form cheese, a milk coagulating enzyme must be added to breakdown the proteins that keep milk a liquid. More particularly, when proteins are denatured or otherwise modified, milk loses its liquid structure and begins to coagulate.

Rennets, milk coagulating enzymes traditionally obtained from the abomasums (the fourth stomach of the calf) have long been used in the production of cheese. The main enzyme extracted from the calf rennet is chymosin. Calf-rennet, however, is expensive and is difficult to obtain due to a chronic shortage of calves to provide chymosin raw materials.

Various milk-coagulating enzymes of animal, plant and microbial origin have been identified as substitutes for chymosin and tested in cheese production. Still, the only-milk-clotting enzymes to be utilized in practice as alternatives for chymosin are pepsin (animal origin) and microbial rennet derived from various types of filamentous fungi, for example Endothia parasitica, Mucor pusillus and Mucor miehei.

U.S. Pat. No. 4,526,792 discloses the use of R. miehei as microbial rennet in the production of cheese. R. miehei does not contain chymosin, but instead acid proteases, which are similar in function to chymosin.

A number of methods to extract and purify milk-coagulating enzymes are known to those skilled in the art. The methods include affinity gel chromatography and subsequent elution of the adsorbed enzymes. For example, Kobayashi, et al., “Rapid isolation of microbial milk-clotting enzymes by N-acetyl-(or N-isobutyryl)-pepstatin-aminohexylagarose” Anal, Biochem., 122: 308-312 (1982) teaches purification of microbial rennet from R. miehei by use of affinity gel column using N-acetylpepstatin as affinity ligand. Enzymes can also be separated on affinity gel columns using Cibacron Blue F3GA (“CB”) as disclosed by Dead, et al., “Protein purification using immobilized triazine dyes,” J. Chromatogr., 165: 301-319 (1979) and Burgett, et al., “Cibacron Blue F3GA affinity chromatography”, Am. Lab., 9(5): 74, 78-83 (1977). Both describe separation of enzymes on CB columns, including for example, kinases and nucleases. U.S. Pat. No. 4,743,551 describes the use of a blue dye affinity ligand and elution of the adsorbed rennet to produce purified R. miehei rennet.

Recent research has been focused on the discovery of a new milk-coagulating enzyme that is a plant derivative and environmental friendly. It has been shown that the leaf extract of Streblus asper (plant Kesinai) contains protease, i.e. a milk coagulating factor, which can be a potential rennet substitute.

Therefore, is advantageous to provide a composition comprising extracts of Streblus asper that is substantially having protease activity.

### SUMMARY OF THE INVENTION

The present invention is directed to a composition comprising a proteinaceous extract of Streblus asper having substantially protease activity that degrades proteins by hydrolysis of peptide bonds.

It is an advantage of the present invention to provide a proteinaceous extract of Streblus asper that is suitable for us a as a meat quality-improving agent and a milk coagulant in food processing industries, as well as an additive in manufacturing of detergents.

### BRIEF DESCRIPTION OF THE DRAWINGS

FIG. 1A is a scanning electron micrograph (SEM) of a proteinaceous extract of Streblus asper (Kesinai) at ×7000;

FIG. 1B is a transmission electron microscopy (TEM) of a proteinaceous extract of Streblus asper (Kesinai)×30,000;

FIG. 2 is a sodium dodecyl sulphate polyacrylamide gel electrophoretic profile (SDS-PAGE) of purified protease;

FIG. 3A is a graph that shows optimum temperature for proteolytic activity of the purified proteas;

FIG. 3B is a graph that shows temperature stability of the purified protease; and

FIG. 4 is a graph that shows the effect of added calcium chloride concentration on milk coagulation time of proteinaceous extract of Streblus asper (Kesinai).

### DETAILED DESCRIPTION OF THE INVENTION

The present invention relates to a proteinaceous leaf extract of plant kesinai, i.e. Streblus asper, which is substantially having protease activity.

Preparation of the crude leaf extract results in an undesirable, very dark brown color and inhibition of this browning may enhance the use of the leaf extract. Browning inhibitors such as citric acid, L-cystein and sodium metabisulphite are used for prevention of browning and to obtain a crude extract with an acceptable color. This solved the main problem of Kesinai leaf extract and enhanced its potential use as a milk coagulant, meat tenderizer and as additive for the detergent industry.

Metabisulphite was found to be an effective inhibitor of the enzymatic browning of the leaf extract. At 2 mM concentration, it inhibited browning and the extract obtained resulted in a white milk coagulum compared to the brown colored coagulum of the brown extract. It is thermostable up to 85° C., with an optimum temperature at 70° C. and its optimum pH is 7.2. 6 mM added calcium chloride was optimum for its milk coagulation activity.

The successful inhibition of the enzymatic browning and characterization of the crude extract makes the basis for examining the physiochemical characteristics of its milk coagulum, purification and characterization of the milk coagulating protease. The use of milk coagulating protease is an essential step in cheese making. Strength, syneresis and yield of the milk coagulum are largely affected by the type of the rennet used. Texture is one of the most important characteristics of cheese, which can be influenced by the type of the coagulant. Textural differences are related to the structural network of the milk coagulum. To study the textural properties, microstructure and syneresis of crude extract is useful in evaluation of the potential suitability of a milk coagulating protease as a rennet substitute. To this extent, milk coagulum was prepared from fresh cow's milk by Streblus asper (kesinai) for scanning electron microscopy (SEM) and transmission electron microscopy (TEM) examinations. These examinations were done to quantify the coagulum porosity, texture and syneresis.

Finally, the crude enzyme extracts from plant kesinai were purified by ultrafiltration (UF), fast protein liquid chromatography (FPLC) gel filtration with Superose 6, FPLC ion exchange using MONOQ HR 5/5 and isoelectric focusing (IEF) using the Rotofor system, with a purification fold of 25, and 18% recovery.

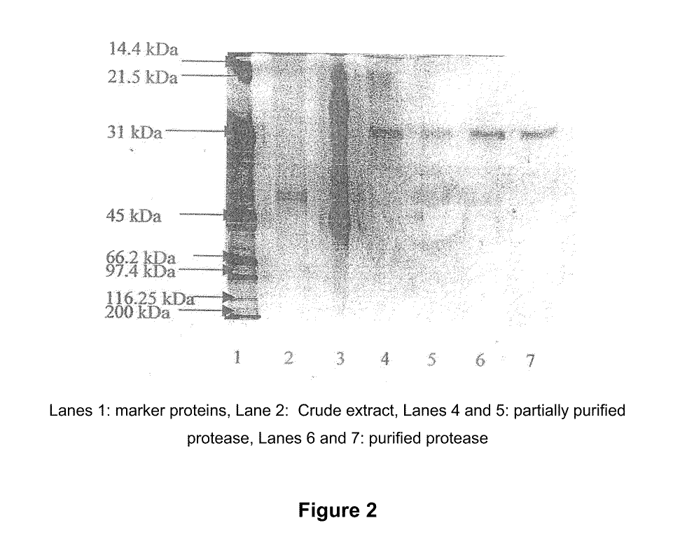

Referring to FIG. 2, the purified protease appeared as a single band on SDS-PAGE with a molecular weight of 31.3 kDa. Characterization of the purified protease showed that it could be a serine protease with optimum pH of 7.2, stable in the pH range 5.0-9.5, and its isoelectric point (pI) is 5.2. It is thermostable up to 85° C., with an optimum temperature of 70° C. Zymogram analysis showed that protease activity is associated with milk coagulation activity. Kesinai protease could be used in the production of short ripened cheese varieties.

The present invention will now be described in greater detail by way of examples, which are not intended limit the scope of the invention.

### Example 1

### Preparation of Leaf Extracts

Fresh Streblus asper leaves were washed and homogenized in 200 ml 100 mM Tris-HCL buffer with pH 6-9 including 0.5-10 mM sodium metabisulphite at room temperature. The homogenate was filtered and centrifuged at 10,000 rpm for 30 minutes at 4° C. The supernatant was collected as crude enzyme extract. Crude enzyme extract was ultrafiltrated and concentrated at room temperature with 43 mm disc membranes using stirred cell Amicon 8050. Retentates and filtrates were collected separately. Then, protease activity was determined using azo-casein in 100 mM Tris-HCL; pH 7.2 as the substrate (0.05%, weight/volume). 100 μl of enzyme was incubated with one ml substrate for one hour at room temperature. The reaction was terminated by the addition of 300 μl trichloroacetic acid. Then, the mixture was centrifuged and the supernatant was collected and its absorbance was measured against a mixture of substrate and buffer as the blank. The change in the absorbance was measured at 410 nano meter and the enzyme activity expressed as 1.0 unit=change of 0.01 absorbance unit.

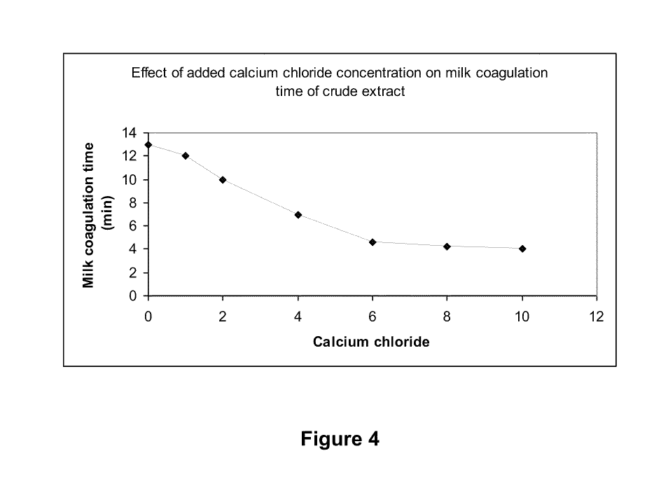

The effect of calcium chloride on milk coagulation time was studied by dissolving calcium chloride in fresh milk to obtain a calcium chloride concentration of 1 to 10 mM. Fresh milk without added calcium chloride was used as control. The milk (2 ml) was tempered for 5 minutes in a water bath at 65° C., then 200 μl crude leaf extract was added. The milk and enzyme mixture was incubated at the set temperature without shaking the water bath. Referring to FIG. 4, the addition of calcium chloride in 1, 2, 4, 6, 8 and 10 mM concentration to fresh milk has increased milk coagulation activity. Milk coagulation activity increased with an increase in added calcium chloride up to a concentration of 6 mM, above which an increase in milk coagulation activity was small.

In this example the effect of sodium metabisulphite for inhibition of enzymatic browning of crude leaf extract was studied. For this reason, the crude extract, prepared by this method, was assayed for color (by measuring the absorbance units with spectrophotometer) and milk coagulation activity.

### Example 2

### Determination of Milk Coagulation Activity

Milk coagulating activity was determined by measuring the time taken by the leaf extract to coagulate 12.5% reconstituted milk. Sample pre-incubated at 65° C. for 5 minutes after which 200 μl leaf extract was added and the mixture was incubated at 65° C. The tube was tilted approximately 45° every 15 seconds. The time taken to form the first visible sign of milk coagulation was recorded as milk coagulation time. One unit milk coagulation activity is that which coagulates 1 ml milk in 1 min under the assay conditions and specific milk coagulation activity is activity unit/mg protein. Boiled enzyme was used as the control.

The result, as shown in Table 1, concludes that a crude extract of an acceptable color was obtained using 10 mM sodium metabisulphite in the extraction buffer. Extract prepared using metabisulphite showed high milk coagulation activity in maintaining protease activity. Sodium metabisulphite is widely used in the food industry as a multifunctional additives and recognized as safe (GRAS) for use as chemical preservation. The level of sulphite used in this study for inhibition of the browning of the leaf extract is low and will not be organoleptically detectable in milk and leaf extract mixture as the level would be ˜38 ppm (part per million) and the minimum threshold for organoleptic detection of sulphite is about 50 ppm (part per million).

The crude leaf extract obtained has an optimum pH of 7.2 and stable in a wide pH range. It is thermostable and has an optimum temperature of 70° C.

TABLE 1
Effect of sodium metabisulphite at various concentrations
on browning of crude leaf extract

| | | | Specific milk |
|---|---|---|---|
| Concentration | Color (absorbance at | Protease specific | coagulation |
| (milli Mole) | 420 nano meter) | activity | activity |
| 0.5 | 1.08 | 13.62 | 0.567 |
| 1.00 | 0.667 | 17.04 | 0.739 |
| 2.00 | 0.519 | 17.22 | 0.754 |
| 3.00 | 0.126 | 17.46 | 0.786 |
| 4.00 | 0.118 | 17.64 | 0.821 |
| 5.00 | 0.114 | 17.70 | 0.836 |
| 10.0 | 0.103 | 17.82 | 0.854 |

### Example 3

### Preparation of Milk Coagulum

For preparing milk coagulum, 0.2 mM calcium chloride and 2 mg of decolorized Streblus asper (kesinai) extract were added to 100 ml fresh cow milk. Then, the mixture was incubated till a coagulum is formed. The prepared coagulum was cut into small pieces and was subjected to scanning electron microscopy (SEM) and transmission electron microscopy (TEM) examinations. Porosity of the milk coagulum was determined by quantification of pores fractional area of SEM and TEM micrographs. For texture, coagulum strength was determined by using a texture analyser.

The extent of syneresis was determined by measuring sample volume and then measuring the volume of whey that could be separated from the coagulum by filtration.

Sodium dodecyl sulphate polyacrylamide gel electrophoresis (SDS-PAGE) was run for the milk coagulum. The results were observed as follow:

The microstructure of the milk coagulum of the leaf extract appeared as a sponge-like when examined under scanning electron microscopy (SEM). The formation of a sponge like structural network by leaf extract was attributed to the nature and proteolytic specificity of the leaf extract in addition to new cross-linkages between casein micelles caused by the phenolic compounds in the leaf extract.

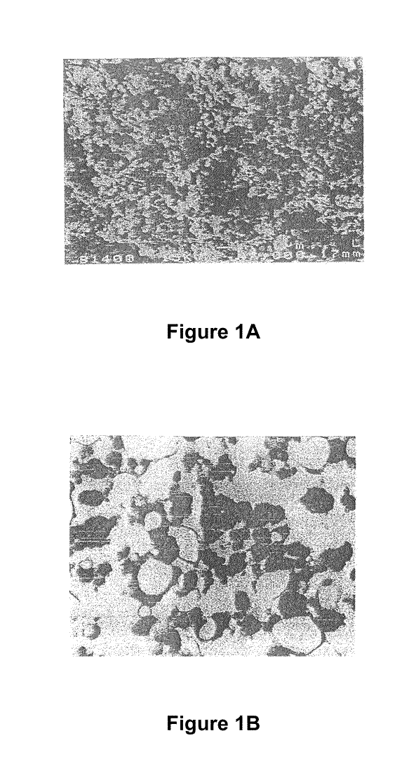

The leaf extract was found to produce a milk coagulum with a lower porosity and a denser casein network. Referring to FIGS. 1a and 1b, both the scanning electron microscopy (SEM) and transmission electron microscopy (TEM) porosity quantification results showed low porosity of kesinai milk coagulum. This is a desirable property in cheese production as casein contribution to cheese yield includes its own weight plus associated moisture and minerals. Also it has expelled less whey, which is the serum phase of milk.

Sodium dodecyl sulphate polyacrylamide gel electrophoretic profile of coagulum and whey (SDS-PAGE) showed that leaf extract has high proteolytic activity.

### Example 4

### Purification and Characterization of Kesinai Milk Coagulating Protease

### (i) Fast Protein Liquid Chromatography (FPLC) Gel Filtration Chromatography

The prepared crude enzyme extracts from the Kesinai leaves with sodium metabisulphite was loaded on a superpose-6 fast protein liquid chromatography (FPLC) column with a bed volume of 25 ml which was equilibrated with 100 mM Tris-HCL; pH 7.2 prior to filtration.

Proteins were eluted with the equilibrating buffer at a flow rate of 0.3 ml per min. Filtration resulted in 4.26 fold purification with a 69.84% yield. The protease containing fractions were pooled and further purified by fast protein liquid chromatography (FPLC) ion exchange chromatography on Mono Q HR 5/5 column. The enzyme was eluted from the column with a salt concentration of 0.35-0.40 M. Ion exchange purification step resulted in 23.76 fold purification with 24.34 percent yield. Protease active fractions eluted from the ion exchange chromatography step were pooled, dialyzed against distilled water and purified by isoelectric focusing the Rotofor apparatus. The 25.10 fold purification was achieved with a final yield of 18 percent.

On the basis of protein, a protein recovery of 140 fold was achieved.

### (ii) Characterization of the Kesinai Protease

Molecular mass determination was estimated using sodium dodecyl sulphate polyacrylamide gel electrophoresis (SDS-PAGE). A standard curve of log molecular mass versus relative mobility of the standard proteins was plotted and the molecular mass of the purified protease was then estimated from the standard curve using its relative mobility.

The results suggest that the protease is probably a monomer consisting of a single subunit. Referring to FIG. 2, electrophoresis experiments to determine molecular mass of the purified protease (lanes 6 and 7) showed a single band with a molecular weight corresponding to about 31.3 kDA. The low molecular weight of the protease is similar to that of serine proteases, which are generally of low molecular weight, usually between 15,000 and 30,000.

Isoelectric point of the purified protease was determined from isoelectric focusing elution profile of the protease, where the protease was eluted as a single peak at pH 5.2, and based on this; its isoelectric point (pI) was estimated to be pH 5.2. Results also showed that the purified protease from Kesinai can coagulate milk even after electrophoresis at pH 8.3 at room temperature.

Optimum activity pH for proteolytic activity of the purified protease over a range of pH values of 5 to 9 for an incubation time of one hour at 37° C. showed a pH optimum for azocasein hydrolysis of 7.2. Under these conditions, the enzyme had 60% of its maximum activity at pH 6.2, which is considered as the pH at which cheese milk is acidified by starter culture.

In order to determine pH stability of the purified protease from kesinai, the enzyme from kesinai was incubated in various pHs from 4.5 to 9.5 for one hour at room temperature. After that, the residual protease activity was determined. Results showed that protease was stable at a pH range of 5 to 8.5 when incubated for one hour at room temperature. In this condition, protease maintained 10% of its activity at pH 5.

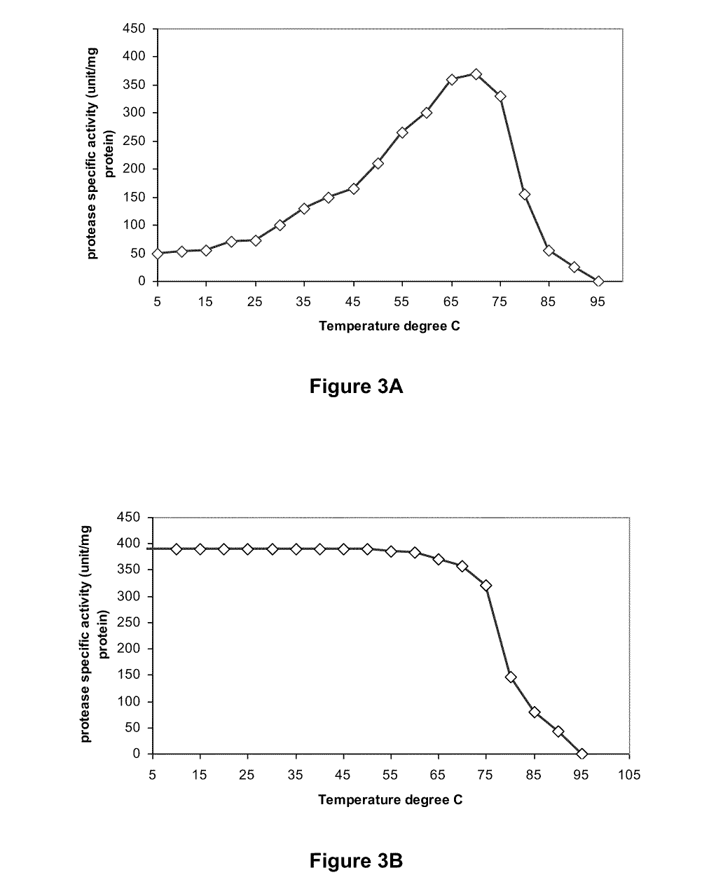

In order to determine the effect of temperature on protease activity, the purified protease was equilibrated for 5 minutes at a temperature ranging from 5 to 95° C. Then, a substrate (azocasein 0.05% w/v in Tris-HCL buffer, pH=7.2) was added and the mixture was incubated at the test temperature for one hour and assayed for proteolytic activity according to standard assay method. Results as shown in FIG. 3A revealed that the optimum temperature of the purified protease was around 70° C.

In order to determine temperature stability of the purified protease from kesinai, the enzyme was incubated at various temperatures in the range of 5 to 95° C. for one hour and then immediately cooled in ice. Residual proteolytic activity was assayed at 37° C. using azocasein (0.05% w/v) as the substrate. The temperature stability of the protease is shown in FIG. 3B. Referring to FIG. 3B, the enzyme was stable up to 75° C. when incubated for one hour. The enzyme activity was 44%, 26% and 14% of its full activity after one hour of incubation at 80° C., 85° C. and 90° C., respectively.

Thermostability of the purified protease indicates that it would be capable of surviving conventional milk and whey pasteurization conditions, which is an undesirable property in rennet substitutes.

### INDUSTRIAL APPLICATION

The crude extract from kesinai according to the present invention can be suitably used as a meat quality-improving agent capable of modifying a meat at an appropriate softness and imparts no undesirable after taste to the meat treated. Moreover, a meat quality-improving agent, the enzyme inactivation temperature of which is relatively low and therefore, which is highly usable for domestic and industrial purposes while easily controlling temperature or inactivation.

Also thermostability and the high proteolytic activity of the crude extract are desirable properties in detergent industry and the purified enzyme could be useful in these processes. Use of enzyme in detergent products can save energy by enabling a lower wash temperature and they are biodegradable, leaving no harmful residues, It will not possess negative environmental impact on sewage treatment processes and also does not present a risk to aquatic life.

The browning of Streblus asper leaf extract indicates that it is rich in phenolic compounds and polyphenoloxidase (PPO), both having potential industrial uses. Polyphenoloxidase is potentially useful in many future industrial applications, including production of flavonoids-derived colorants as antioxidants and the removal of oestrogenic substances from aquatic environments. Being a rich source of phenolic compounds, Streblus asper leaf extract could be useful in improving the thermal and colloidal stability of concentrated milk, as new evident suggests that plant extracts rich in phenolic compounds markedly increase the heat and colloidal stability of milk.

While the illustrative embodiments of the invention have been described with particularly, it will be understood that various other modifications will be apparent to and can be readily made by those skilled in the art without departing from the scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A composition comprising a proteinaceous extract of Streblus asper substantially having protease activity.

2. A composition according to claim 1, wherein the proteinaceous extract of Streblus asper has a molecular weight of 31.3 kDA

3. A composition according to claim 1, wherein the proteinaceous extract of Streblus asper has an isoelectric point of pH 5.2.

4. A composition according to claim 1, wherein the proteinaceous extract of Streblus asper has a protease activity within a pH range of 5 to 9.

5. A composition according to claim 1, wherein the proteinaceous extract of Streblus asper has a protease stability within a pH range of 5 to 8.5

6. A composition according to claim 1, wherein the proteinaceous extract of Streblus asper has an optimum temperature of activity of 70° C.
